(19) United States
(12) Patent Application Publication
(10) Pub. No.: US 2004/0266652 A1
(43) Pub. Date: Dec. 30, 2004

(54) Nonionic surfactant compositions

(76) Inventors: David W. Brown, Ambler, PA (US); Kenneth Breindel, Lansdale, PA (US); Ronald W. Broadbent, Horsham, PA (US); Michael S. Wiggins, Lansdale, PA (US)

Correspondence Address: COGNIS CORPORATION, PATENT DEPARTMENT, 300 BROOKSIDE AVENUE, AMBLER, PA, 19002, US

(21) Appl. No.: 10/855,545

(22) Filed: May 27, 2004

Related U.S. Application Data
(60) Provisional application No. 60/474,012, filed on May 29, 2003.

(51) Int. Cl. A61K 31/40

(52) U.S. Cl. 510/470

(57) ABSTRACT

Surfactant concentrates containing the following components:

I) at least one base-catalyzed reaction product of reactants comprising:

A) at least one compound of formula I

R¹(X)₃ (I)

wherein each X group is a halogen atom or one X group is a halogen atom and two X groups represent an epoxy oxygen atom, which is attached to two adjacent carbon atoms in the R¹ group to form an epoxy group, and R¹ is an alkanetriyl group containing from 3 to 10 carbon atoms; and

B) at least one compound having the formula II

R²X(AO)ₙY (II)

wherein R² is a substituted or unsubstituted, saturated or unsaturated, organic group having from 1 to 36 carbon atoms; X is —O—, —S—, or —NR³— where R³ is hydrogen or a C₁-C₁₈ alkyl group; each AO group is independently an ethyleneoxy, 1,2-propyleneoxy, or 1,2-butyleneoxy group, n is a number of from 0 to 200, preferably from 1 to 100, more preferably from 2 to 20; and Y is hydrogen, or Y can be a mercapto group or an amino group (amino or C₁-C₆ alkylamino group) in place of the terminal —OH group, provided that when Y is mercapto, amino, or a C₁-C₆ alkylamino group, n is at least 1; and

II) at least one nonionic surfactant other than nonionic surfactants of component I).

### CROSS-REFERENCE TO RELATED APPLICATIONS

This application claims priority, under 35 U.S.C. §119(e), of U.S. Provisional Patent Application Ser. No. 60/474,012, filed on May 29, 2003, the entire contents of which are hereby incorporated by reference.

### BACKGROUND OF THE INVENTION

Surfactant concentrates are well known in the art. Such concentrates are economical to ship to formulators who dilute the concentrates with water or add the concentrates to cleaning compositions for use as a component of dishwasher detergents, laundry detergents, all purpose cleaners including spray cleaners and the like.

A recurring problem with surfactant concentrates, including nonionic surfactant-containing concentrates, is to obtain single phase, uniform, and stable concentrates of two or more surfactants having more than a 5 to 10 times concentration, since incompatibility and insufficient solubility of the surfactant mixtures can prevent the successful formulation of such highly concentrated surfactant compositions.

### BRIEF SUMMARY OF THE INVENTION

The present invention relates to surfactant compositions in the form of aqueous concentrates.

It has now been discovered that nonionic surfactant-containing concentrate mixtures that contain, as one surfactant component thereof, at least one base-catalyzed reaction product of reactants comprising:

A) at least one compound of formula I

R¹(X)₃ (I)

wherein each X group is a halogen atom or one X group is a halogen atom and two X groups represent an epoxy oxygen atom, which is attached to two adjacent carbon atoms in the R¹ group to form an epoxy group, and R¹ is an alkanetriyl group containing from 3 to 10 carbon atoms; and

B) at least one compound having the formula II

R²X(AO)ₙY (II)

wherein R² is a substituted or unsubstituted, saturated or unsaturated, organic group having from 1 to 36 carbon atoms; X is —O—, —S—, or —NR³— where R³ is hydrogen or a C₁-C₁₈ alkyl group; each AO group is independently an ethyleneoxy, 1,2-propyleneoxy, or 1,2-butyleneoxy group, n is a number of from 0 to 200, preferably from 1 to 100, more preferably from 2 to 20; and Y is hydrogen, or Y can be a mercapto group or an amino group (amino or C₁-C₆ alkylamino group) in place of the terminal —OH group, provided that when Y is mercapto, amino, or a C₁-C₆ alkylamino group, n is at least 1;

result in concentrates that can be highly concentrated, e.g. to from 15 to 50 times, wherein the concentrates are single phase, uniform, and stable free flowing liquids, and can be readily diluted with water without any tendency to gel.

### DETAILED DESCRIPTION OF THE INVENTION

Other than in the operating examples, or where otherwise indicated, all numbers expressing quantities of ingredients or reaction conditions used herein are to be understood as modified in all instances by the term “about”.

The aqueous surfactant concentrates of the invention comprise the following components:

I) at least one base-catalyzed reaction product of reactants comprising:

A) at least one compound of formula I

R¹(X)₃ (I)

wherein each X group is a halogen atom or one X group is a halogen atom and two X groups represent an epoxy oxygen atom, which is attached to two adjacent carbon atoms in the R¹ group to form an epoxy group, and R¹ is an alkanetriyl group containing from 3 to 10 carbon atoms; and

B) at least one compound having the formula II

R²X(AO)ₙY (II)

wherein R² is a substituted or unsubstituted, saturated or unsaturated, organic group having from 1 to 36 carbon atoms; X is —O—, —S—, or —NR³— where R³ is hydrogen or a C₁-C₁₈ alkyl group; each AO group is independently an ethyleneoxy, 1,2-propyleneoxy, or 1,2-butyleneoxy group, n is a number of from 0 to 200, preferably from 1 to 100, more preferably from 2 to 20; and Y is hydrogen, or Y can be a mercapto group or an amino group (amino or C₁-C₆ alkylamino group) in place of the terminal —OH group, provided that when Y is mercapto, amino, or a C₁-C₆ alkylamino group, n is at least 1; and

II) at least one nonionic surfactant other than nonionic surfactants of component I).

The weight ratio of component I) to component II) is in the range of from 1:99 to 99:1, preferably from 10:90 to 90:10, more preferably from 10:90 to 25:75, and most preferably from 15:85 to 25:75.

The solids content (nonaqueous components) of the aqueous concentrates is from 25 to 60% by weight, preferably from 40 to 50% by weight, with the remainder preferably all water.

In component I) above, the mole ratio of the linking compounds A) to B) is from 0.1:1 to 5:1, preferably from 0.6:1 to 2:1, more preferably from 0.8:1 to 2:1, and most preferably from 1.0:1 to 1.5:1.

In the above compounds of component A), the linking compound of formula I is preferably epichlorohydrin or another epihalohydrin. Also, trihaloalkanes can be used, such as 1,2,3-trichloropropane, 1,2,4-trichlorobutane, 1,3,6-trichlorohexane, and the like. Instead of chlorine in the epichlorohydrins and the trihaloalkanes, the corresponding bromine and iodine compounds can also be used, including compounds containing two or even all three of the above halogens.

The component B) compounds of formula II are organic (optionally alkoxylated) alcohols or the corresponding sulfhydryl or amine compounds.

The R² group can be a substituted or unsubstituted, saturated or unsaturated hydrocarbon group having from 1 to 36 carbon atoms. Examples of such hydrocarbon groups include linear or branched alkyl groups having from 1 to 36 carbon atoms, preferably from 4 to 22 carbon atoms, linear or branched alkenyl or alkynyl groups having from 2 to 36 carbon atoms, preferably from 4 to 22 carbon atoms, aryl groups having from 6 to 22 carbon atoms, and arenyl groups having from 7 to 36 carbon atoms. Arenyl groups are alkyl-substituted aromatic radicals having a free valance at an alkyl carbon atom such as a benzylic group.

The R² group can also be a saturated carbocyclic group, an unsaturated carbocyclic group having one or more multiple bonds, a saturated heterocyclic group, or an unsaturated heterocyclic group having one or more multiple bonds. Any of the above R² groups can be substituted groups, i.e. the groups can be single or multiple substituents such as one or more halogen substituents, for example, Cl, Fl, I, and Br; a sulfur functionality such as a mercaptan or thio group, a nitrogen functionality such as an amine or amide functionality; an alcohol functionality, a silicon functionality, e.g., a siloxane; an ether functionality, e.g. a C₁-C₆ alkoxy group; or any combination thereof.

The R² group in formula II is preferably a branched chain alkyl group containing from 4 to 36 carbon atoms, preferably from 4 to 12 carbon atoms, and more preferably from 8 to 10 carbon atoms.

When the X group of formula II is an —S— group, the R² group will preferably have from about 4 to about 22 carbon atoms, examples of which include but are not limited to, dodecyl mercapto and 1-hexadecanethio.

When the R²X-group of formula II is a secondary or tertiary amino group, the group preferably contains from 4 to 22 carbon atoms, and n is preferably a number of from 4 to 50. Examples of primary and secondary amines useful for obtaining the R²X-group include, but are not limited to, dibutyl amine, cyclohexyl amine, isodecyl amine, and dioctylamine.

When Y in formula II is an amine or sulfhydryl group, the resulting compounds can be readily prepared from the corresponding alcohols wherein the terminal hydroxy group is replaced by an —SH group or by an amine nitrogen. For example, a compound of formula II where Y is —OH can be subjected to a catalyzed ammoniation (with ammonia, or a lower alkylamine) for replacement of the hydroxyl.

In the compounds of formula II, the AO groups when present are preferably all ethyleneoxy groups. However, as stated above, each AO group can be independently an ethyleneoxy (EO), 1,2-propyleneoxy (PO), or 1,2-butyleneoxy (BO) group, i.e. any one or more of such groups can be present, and they can be present in any order, as well as be present in blocks, e.g. compounds of formula III:

R²O(EO)ₘ(PO)ₚ(BO)_{q}H (III)

wherein R² has the meaning given above, m is a number of from 0 to 100, preferably from 1 to 50, p is a number of from 0 to 50, and q is a number of from 0 to 50. Compounds of formula III in which R² is a branched chain alkyl group having from 4 to 12 carbon atoms, m is a number of from 2 to 20, and p and q are 0 are preferred. The most preferred compound is the reaction product of epichlorohydrin and isodecyl alcohol·4EO, marketed by Cognis Corporation Ambler, Pa., 19002 as DEHYPOUND® ST-15.

The degree of hydrophilic and hydrophobic properties of the reaction products of components A) and B) can be readily controlled by controlling the type and number of alkyleneoxy groups in component B) and the number of carbon atoms in the R² group. For example, the greater the number of ethyleneoxy groups present, the greater the water solubility, while the presence of 1,2-propyleneoxy groups and/or 1,2-butyleneoxy groups for example, will decrease water solubility.

In general, the compounds of formula III wherein the sum of n, m, and p is at least 1, and especially at least 2 are preferred for use herein.

The above reaction products can be prepared by the process disclosed in U.S. Pat. No. 5,827,453, the disclosure of which is expressly incorporated herein by reference.

In general, the component A) and B) reactants are reacted together, preferably in the presence of an inert organic solvent such as toluene that will azeotrope water, and in the presence of a base, such as an aqueous sodium hydroxide, at a temperature of from 60° to 125° C. Preferably component B) is first mixed with the base and the organic solvent, and water is removed by azeotropic distillation. Then component A) is slowly added and the reaction continued until the reaction is completed. The reaction mixture is filtered and vacuum stripped to remove the organic solvent.

Component II) can be any water soluble nonionic or polar nonionic surface active agent or a mixture of two or more of such surface active agents.

Preferred surfactants of component II are alkyl polyglycosides having formula IV below:

R₁O(R₂O)_{b}(Z)ₐ (IV)

wherein R₁ is a monovalent organic radical having from 6 to 30 carbon atoms, preferably from 6 to 16 carbon atoms; R₂ is a divalent alkylene radical having from 2 to 4 carbon atoms; Z is a saccarhide residue having 5 or 6 carbon atoms; b is a number having a value from 0 to 12; a is a number having a value from 1 to 6, preferably from 1.2 to 2.2, and more preferably from 1.4 to 1.7. Preferred alkyl polyglycosides which can be used in the compositions according to the invention have the formula I wherein Z is a glucose residue and b is zero. Such alkyl polyglycosides are commercially available, for example, as APG®, GLUCOPON®, or PLANTAREN® surfactants from Cognis Corporation, Ambler, Pa., 19002. Examples of such surfactants include but are not limited to:

1. GLUCOPON® 225 DK Surfactant—an alkyl polyglycoside in which the alkyl group contains 8 to 10 carbon atoms and having an average degree of polymerization of 1.7.

2. GLUCOPON® 425N Surfactant—an alkyl polyglycoside in which the alkyl group contains 8 to 16 carbon atoms, having an average of 10.3 carbon atoms, and having an average degree of polymerization of 1.5.

3. GLUCOPON® 625 UP Surfactant—an alkyl polyglycoside in which the alkyl group contains 12 to 16 carbon atoms and having an average degree of polymerization of 1.6.

4. APG® 325N Surfactant—an alkyl polyglycoside in which the alkyl group contain 9 to 11 carbon atoms and having an average degree of polymerization of 1.5.

5. GLUCOPON® 600 UP Surfactant—an alkyl polyglycoside in which the alkyl group contains 12 to 16 carbon atoms and having an average degree of polymerization of 1.4.

6. PLANTAREN® 2000 Surfactant—a C₈-C₁₆ alkyl polyglycoside in which the alkyl group contains 8 to 16 carbon atoms and having an average degree of polymerization of 1.5.

7. PLANTAREN® 1300 Surfactant—a C₁₂-C₁₆ alkyl polyglycoside in which the alkyl group contains 12 to 16 carbon atoms and having an average degree of polymerization of 1.6.

8. GLUCOPON® 220N Surfactant—an alkyl polyglycoside in which the alkyl group contains 8 to 10 carbon atoms and having an average degree of polymerization of 1.5.

Other examples include alkyl polyglycoside surfactant compositions which are comprised of mixtures of compounds of formula I wherein Z represents a moiety derived from a reducing saccharide containing 5 or 6 carbon atoms; a is a number having a value from 1 to about 6; b is zero; and R₁ is an alkyl radical having from 8 to 20 carbon atoms. The compositions are characterized in that they have increased surfactant properties and an HLB in the range of about 10 to about 16 and a non-Flory distribution of glycosides, which is comprised of a mixture of an alkyl monoglycoside and a mixture of alkyl polyglycosides having varying degrees of polymerization of 2 and higher in progressively decreasing amounts, in which the amount by weight of polyglycoside having a degree of polymerization of 2 or mixtures thereof with the polyglycoside having a degree of polymerization of 3 predominate in relation to the amount of monoglycoside, said composition having an average degree of polymerization of about 1.8 to about 3. Such compositions, also known as peaked alkyl polyglycosides, can be prepared by separation of the monoglycoside from the original reaction mixture of alkyl monoglycoside and alkyl polyglycosides after removal of the alcohol. This separation may be carried out by molecular distillation and normally results in the removal of about 70-95% by weight of the alkyl monoglycosides. After removal of the alkyl monoglycosides, the relative distribution of the various components, mono- and poly-glycosides, in the resulting product changes and the concentration in the product of the polyglycosides relative to the monoglycoside increases as well as the concentration of individual polyglycosides to the total, i.e. DP2 and DP3 fractions in relation to the sum of all DP fractions. Such compositions are disclosed in U.S. Pat. No. 5,266,690, the entire contents of which are incorporated herein by reference.

The most preferred alkyl polyglycosides for use in the present invention are GLUCOPON® 425 or 425N, which are alkyl polyglycosides in which the alkyl group contains an average of 10.3 carbon atoms, and the average degree of polymerization is 1.5.

Other nonionic surfactants that can be used alone or in mixtures as component II include the condensation product of an organic aliphatic or alkyl aromatic hydrophobic compound and hydrophilic ethylene oxide groups. Practically any hydrophobic compound having a carboxy, hydroxy, amido, or amino group with a free hydrogen attached to the nitrogen can be condensed with ethylene oxide or with the polyhydration product thereof, polyethylene glycol, to form a nonionic surfactant. Further, the length of the polyethyleneoxy chain can be adjusted to achieve the desired balance between the hydrophobic and hydrophilic elements.

The nonionic surfactants of component II) also include the polyethylene oxide condensate of one mole of alkyl phenol containing from about 6 to 12 carbon atoms in a straight- or branched-chain configuration with about 5 to 30 moles of ethylene oxide, e.g., nonyl phenol condensed with 9 moles of ethylene oxide, dodecyl phenol condensed with 15 moles of ethylene oxide and dinonyl phenol condensed with 15 moles of ethylene oxide. Condensation products of the corresponding alkyl thiophenols with 5 to 30 moles of ethylene oxide are also suitable.

Still other suitable nonionics are the polyoxyethylene polyoxypropylene adducts of 1-butanol. The hydrophobe of these nonionics has a minimum molecular weight of 1,000 and consists of an aliphatic monohydric alcohol containing from 1 to 8 carbon atoms to which is attached a chain of oxyethylene and oxypropylene. The weight ratio of oxypropylene to oxyethylene covers the range of 95:5 to 85:15. Attached to this is the hydrophilic polyoxyethylene chain which is from 44.4 to 54.6 percent of the total molecular weight of 1,400 to 4,000.

Also, included are the condensation products of a higher alcohol containing about 8 to 18 carbon atoms in a straight or branched-chain configuration condensed with about 5 to 30 moles of ethylene oxide, e.g., lauryl-myristyl alcohol condensed with about 16 moles of ethylene oxide.

Other suitable nonionics may be derived by the condensation of ethylene oxide with the product resulting from the reaction of propylene oxide and ethylene diamine. The molecular weight varies from 500 to 4,500 or more.

The polar nonionic surfactants that can be used as component II) include open-chain aliphatic amine oxides of the general formula R₁R₂R₃N→O. For the purposes of this invention R₁ is an alkyl, alkenyl, or monohydroxyalkyl radical having about 10 to 16 carbon atoms. R₂ and R₃ are each selected from the group consisting of hydrogen, methyl, ethyl, propyl ethanol, and propanol radicals.

Other operable polar nonionic detergents are the open-chain aliphatic phosphine oxides having the general formula R₁R₂R₃P→O wherein R₁ is an alkyl, alkenyl, or monohydroxyalkyl radical ranging in chain length from 10 to 18 carbon atoms, and R₂ and R₃ are each alkyl and monohydroxyalkyl radicals containing from 1 to 3 carbon atoms.

The concentrates of the invention provide reduced storage and shipping space and expense, since they are highly concentrated. In addition, they contain no volatile organic solvents and hence present no fire or explosion risks.

As discussed above, preferred concentrates of the invention contain only components I) and II) in addition to water, although small quantities of other components such as antimicrobial agents, perfumes, and the like can also be present.

The above concentrates can be readily diluted with water to obtain in-use dilutions, e.g. by adding 1 part of the concentrate to from 25 to 60 parts by weight of water to produce a low foaming, non-gelling surfactant composition, or the concentrates can be added to cleaning excipients and components such as builders, alkaline agents, inorganic salts, bleaching agents, alcohol sulfates, fluorescent whitening agents, antimicrobial agents, antiredeposition agents, blueing agents, proteolytic enzymes, organic sequestering agents, and the like to form liquid cleaning compositions or concentrates.

The present invention will now be illustrated in more detail by reference to the following specific, non-limiting examples.

### EXAMPLE 1

A free flowing liquid highly concentrated concentrate was prepared by mixing together the following components:

| Component | % by weight | % Active |
|---|---|---|
| Water | 31.50 | — |
| GLUCOPON ® 425N¹ | 61.65 | 30.83 |
| DEHYPOUND ® ST-15² | 6.85 | 6.85 |
| | 100.0 | 37.68 |

¹GLUCOPON ® 425N - an alkyl polyglycoside in which the alkyl group contains an average of 10.3 carbon atoms and an average degree of polymerization of 1.5.
²DEHYPOUND ® ST-15 - the base-catalyzed reaction product of epichlorohydrin and isodecyl alcohol.4EO, produced by the following process:

About 150 grams of decyl alcohol ethoxylated with an average of 4 moles of ethylene oxide (0.45 OH equivalents) were mixed with 385 grams of toluene and 54 grams of 50% aq. NaOH (0.675 equivalents). The water was removed by azeotropic distillation and when a moisture level of less than 0.8% was reached, about 46 grams (0.51 equivalents) of epichlorohydrin were slowly added. This mixture was allowed to react at 100°-110° C. for 24 hours. An aliquot of this mixture was removed and filtered to remove the NaCl and vacuum stripped to remove the toluene to give an amber, easily pourable liquid product that was dispersible in water.

### EXAMPLE 2

The following concentrate was prepared by mixing together the following components:

| Component | % by weight | % Active |
|---|---|---|
| GLUCOPON ® 425N | 64.8 | 32.4 |
| DEHYPOUND ® ST-15 | 7.2 | 7.2 |
| Water | 28.0 | — |
| | 100.0 | 39.6 |

### EXAMPLE 3

A free flowing liquid highly concentrated organic solvent-free all-purpose spray cleaner concentrate was prepared by mixing together the following components:

| Component | % by weight | % Active |
|---|---|---|
| Water* | 17.00 | — |
| potassium hydroxide (45%) | 6.00 | 2.70 |
| sodium citrate .2H₂O | 7.50 | 6.58 |
| GLUCOPON ® 425N | 61.65 | 30.83 |
| DEHYPOUND ® ST-15 | 6.85 | 6.85 |
| | 100.0 | 46.96 |

*total water is 53.04% based on 46.96% actives

### EXAMPLE 4

The following volatile organic compound-free all purpose cleaner concentrate was prepared by mixing together the following components:

| Component | % by weight | % Active |
|---|---|---|
| GLUCOPON ® 425N | 64.8 | 32.4 |
| DEHYPOUND ® ST-15 | 7.2 | 7.2 |
| Triethanolamine | 10.0 | 10.0 |
| Tetrasodium EDTA | 15.0 | 5.85 |
| SULFOTEX ® 110* | 3.0 | 0.93 |
| Total water content | 43.62 | — |

*SULFOTEX ® 110 is a 31% active solution of n-decyl alcohol sulfate, sodium salt. The above concentrate had a specific gravity of 1.130 and a pH of 11.8.

### EXAMPLE 5

An aqueous nonionic surfactant concentrate is prepared by mixing together the following components:

| Component | % by weight |
|---|---|
| Water | 60.0 |
| DEHYPOUND ® ST-15 | 10.0 |
| Lauryl-myristyl alcohol .30EO | 30.0 |

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

What is claimed is:

1. A liquid surfactant concentrate comprising:
I) at least one base-catalyzed reaction product prepared by a process which comprises reacting:
A) at least one compound of formula I
R¹(X)₃ (I)
wherein each X group is a halogen atom or one X group is a halogen atom and two X groups represent an epoxy oxygen atom, which is attached to two adjacent carbon atoms in the R¹ group to form an epoxy group, and R¹ is an alkanetriyl group containing from 3 to 10 carbon atoms; and
B) at least one compound having the formula II
R²X(AO)ₙY (II)
wherein R² is a substituted or unsubstituted, saturated or unsaturated, organic group having from 1 to 36 carbon atoms; X is —O—, —S—, or —NR³— where R³ is hydrogen or a C₁-C₁₈ alkyl group; each AO group is independently an ethyleneoxy, 1,2-propyleneoxy, or 1,2-butyleneoxy group, n is a number of from 0 to 200, preferably from 1 to 100, more preferably from 2 to 20; and Y is hydrogen, or Y can be a mercapto group or an amino group or a C₁-C₆ alkylamino group in place of the terminal —OH group, provided that when Y is mercapto, amino, or a C₁-C₆ alkylamino group, n is at least 1; and
II) at least one nonionic surfactant other than nonionic surfactants of component I).

2. The concentrate of claim 1 wherein the weight ratio of component I) to component II) is from about 1:99 to about 99:1.

3. The concentrate of claim 2 wherein said ratio is from about 10:90 to about 90:10.

4. The concentrate of claim 2 wherein said ratio is from about 10:90 to about 25:75.

5. The concentrate of claim 2 wherein said ratio is from about 15:85 to about 25:75.

6. The concentrate of claim 1 wherein the nonaqueous components of the concentrate is in the range of from about 25 to about 60% by weight.

7. The concentrate of claim 6 wherein said range is from about 40 to about 50% by weight.

8. The concentrate of claim 1 wherein in component I) the mole ratio of component A) to component B) is from about 0.1:1 to about 5:1.

9. The concentrate of claim 8 wherein the mole ratio is from about 0.6:1 to about 2:1.

10. The concentrate of claim 8 wherein said mole ratio is from about 0.8:1 to about 2:1.

11. The concentrate of claim 8 wherein said mole ratio is from about 1.0:1 to about 1.5:1.

12. The concentrate of claim 1 wherein component II) is selected from the group consisting of water soluble nonionic surfactants and water soluble polar nonionic surfactants.

13. The concentrate of claim 1 wherein component II) is at least one alkyl polyglycoside.

14. The concentrate of claim 13 wherein the at least one alkyl polyglycoside has the following formula:
R₁O(R₂O)_{b}(Z)ₐ (IV)
wherein R₁ is a monovalent organic radical having from about 6 to about 30 carbon atoms; R₂ is a divalent alkylene radical having from 2 to 4 carbon atoms; Z is a saccarhide residue having 5 or 6 carbon atoms; b is a number having a value from 0 to about 12; and a is a number having a value from 1 to about 6.

15. The concentrate of claim 4 wherein in formula IV, Z is a glucose residue and b is zero.

16. The concentrate of claim 1 wherein component I)A) is epichlorohydrin.

17. The concentrate of claim 1 wherein in formula II, n is a number of from 1 to 100.

18. The concentrate of claim 17 wherein n is a number of from 2 to 20.

19. The concentrate of claim 1 wherein in component I)A), the R² group is a straight or branched chain alkyl group.

20. The concentrate of claim 19 wherein in component I)A), n is a number from 2 to 20.

21. The concentrate of claim 1 wherein component I)A) has the formula:
R²O(EO)ₘ(PO)ₚ(BO)_{q}H (III)
wherein R² has the meaning given in claim 1, m is a number of from 0 to 100, p is a number of from 0 to 50, and q is a number of from 0 to 50.

22. The concentrate of claim 21 wherein component A) is epichlorohydrin.

23. The concentrate of claim 21 wherein the mole ratio of component A) to component B) is from about 0.1:1 to about 5:1.

24. The concentrate of claim 23 wherein said mole ratio is from about 0.8:1 to about 2:1.

25. The concentrate of claim 23 wherein said mole ratio is from about 1.0:1 to about 1.5:1.

26. The concentrate of claim 21 wherein m is a number of from 2 to 20.

27. The concentrate of claim 26 wherein p and q=0.

28. The concentrate of claim 23 wherein R² is an alkyl group having from 4 to 12 carbon atoms.

29. The concentrate of claim 21 wherein R² is an alkyl group having from 4 to 12 carbon atoms, m is a number of from 4 to 50, and p and q=0.

30. The concentrate of claim 1 wherein component I) is the reaction product of isodecyl alcohol·4EO and epichlorohydrin and component II) is an alkyl polyglycoside wherein the alkyl group has an average of from about 10 to about 10.5 carbon atoms and an average degree of polymerization of from 1.5 to 1.7.

31. The concentrate of claim 30 wherein in component I)B) the alkyl group has an average of about 10.3 carbon atoms and the average degree of polymerization is about 1.6.

32. The concentrate of claim 1 wherein component I)A) has the formula:
R²O(EO)ₘ(PO)ₚ(BO)_{q}H (III)
wherein R² has the meaning given in claim 1, m is a number of from 0 to 100, p is a number of from 0 to 50, and q is a number of from 0 to 50; and wherein component II) has the formula:
R₁O(R₂O)_{b}(Z)ₐ (IV)
wherein R₁ is a monovalent organic radical having from about 6 to about 30 carbon atoms; R₂ is a divalent alkylene radical having from 2 to 4 carbon atoms; Z is a saccarhide residue having 5 or 6 carbon atoms; b is a number having a value from 0 to about 12; and a is a number having a value from 1 to about 6.
